(19) United States
(12) Patent Application Publication
(10) Pub. No.: US 2023/0277712 A1
(43) Pub. Date: Sep. 7, 2023

(54) DEVICE TO GENERATE REACTIVE OXYGEN SPECIES (ROS) AND METHOD THEREOF

(71) Applicant: GEORGE MASON UNIVERSITY, Fairfax, VA (US)

(72) Inventors: Pei Dong, Fairfax, VA (US); Qiliang Li, Fairfax, VA (US); Xiaozhou Huang, Manassas, VA (US); Zhe Wang, Rochester, MI (US)

(21) Appl. No.: 18/177,305

(22) Filed: Mar. 2, 2023

Related U.S. Application Data
(60) Provisional application No. 63/315,618, filed on Mar. 2, 2022.

(51) Int. Cl. A61L 9/12; B82Y 30/00

(52) U.S. Cl. CPC A61L 9/12; B82Y 30/00

(57) ABSTRACT

An embodiment of the invention provides a composition comprising a carbon material and an ionic liquid (IL), wherein the composition is configured to form a reactive oxygen species (ROS) in presence of oxygen and a radiation having wavelength in a range of about 150 nm to about 1100 nm. In another embodiment, the invention provides a method to generate ROS using the ionic liquid (IL) and the carbon material and the method to measure the same in situ.

### RELATED APPLICATION

This application claims priority from U.S. provisional application 63/315,618 titled as “DEVICE TO GENERATE REACTIVE OXYGEN SPECIES (ROS) AND METHOD THEREOF” filed on Mar. 2, 2022, which is incorporated herein by reference in its entirety.

### FIELD OF THE INVENTION

This invention relates to a system and a process of preparing reactive oxygen species. The invention is more particularly concerned with a process involving use of a carbon material for the production of the reactive oxygen species.

### BACKGROUND OF INVENTION

Zhang et al. 2020 Advanced Science, 8(3), 2002797 states “Reactive oxygen species (ROS) is a general term used to describe the species of highly active radicals formed upon unpaired electrons of oxygen such as hydroxyl radical (·OH) and superoxide (·O₂⁻). The term ROS is most often expanded to include reactive oxygen-containing compounds or nonradical oxidizing agents such as singlet oxygen (¹O₂), ozone (O₃), hydrogen peroxide (H₂O₂), and hypochlorous acid (HOCl).”

Plenty of ROS-based disinfection technologies have been developed for self-cleaning membranes and protein deactivation regulations in the lab to kill viruses, bacteria, or deactivate proteins; however, in practice, generating ROS in aqueous media comes with low yield, high cost, fast decay, difficult storage and unclear generation mechanisms that result from the competitive reactions among ROS and other byproducts and side reactions in the water.

For instance, ¹O₂ only lasts for 4 μs in water. The lifetime of O²⁻ is also very short, only about 1 min if existing in basic aqueous solution. Tain, R. W. et al. 2018 Journal of Magnetic Resonance Imaging, 47(1), 222-229 states, “the lifetime of singlet oxygen, superoxide, and hydroxyl radical in aqueous solutions is within the μs regime.”

ROS can quickly react with hydrogen ions (protons) in the environment to form stable substances. For example, O²⁻ reacts with hydrogen to produce H₂O₂. Meanwhile, the HO· radical can be consumed by organic compounds, carbonates, or other ROS quenchers within a few microseconds [26]. The combination of these factors has thus far limited the kinetic studies of the generation process and its applications. This remains a major challenge in further designs of energy-efficient systems which yield high concentrations of ROS and are able to sustain long-term use.

Thus, it is important to establish a new research environment to find a more efficient method for ROS generation which utilizes green processes and feedstock and ensures that the ROS produced are sufficient for long-term use.

### SUMMARY OF INVENTION

The invention relates to a new concept of generation mechanisms for ROS, developing a new ROS storage method and deriving a disinfection technology for public health.

Compared with the existing public disinfection methods, the main improvements of the present invention are supposed to be: 1) more efficient and stronger disinfection performance, 2) suitable to disinfect a wide range of substances such as virus, bacterial, volatile organic compounds, hazard/toxic gases 3) low-cost and high reusability, 4) long duration usage and storage for the disinfecting substances, 5) easy-to-use, 6) odorless and 7) less harmful or toxic to humans.

In an embodiment, ROS is a type of highly reactive substance that can be generated from carbon materials when illuminated with a solar radiation.

An embodiment relates to generation of a ROS from SWCNTs in a non-aqueous ionic liquid (IL) to form a suspension system for generating superoxide (O₂⁻) under UV light to near infra-red light.

An embodiment of the invention relates to a disinfection technology that can efficiently generate disinfectants, reactive oxygen species (ROS), by using carbon materials to kill most of bacteria or virus (such as COVID-19) and ensure cleanliness and safety for public spaces in the daytime and at night for a long period of time. This platform technology could effectively reduce the toxicity level caused by organic/inorganic small molecules, bacteria, and virus.

In an embodiment, the invention provides a composition comprising a carbon material and an ionic liquid (IL), wherein the composition is configured to form a reactive oxygen species (ROS) in presence of oxygen and a radiation having wavelength in a range of about 150 nm to about 1100 nm.

In an embodiment, ROS is superoxide.

In an embodiment, about 0.001% w/v to about 1% w/v of the carbon material is suspended in the IL.

In an embodiment, the carbon material is suspended in the ionic liquid for a predetermined time ranging from 0 min to 36 hours before irradiation of the radiation.

In an embodiment, the radiation having a wavelength in the range of about 250 nm to 500 nm.

In an embodiment, about 85% of superoxide generated by the composition has a stability for at least 75 hours in the IL.

In an embodiment, a half lifetime of the ROS in the IL is up to 200 hours.

In an embodiment, a half lifetime of the superoxide in the IL is up to 200 hours.

In an embodiment, a half lifetime of the superoxide in the IL is between 24 hours to 200 hours.

In an embodiment, generation of the superoxide in the composition is an irreversible process.

In an embodiment, a ROS generation efficiency of the composition is in a range of about 2.5*10⁻⁴ mol L⁻¹g⁻¹s⁻¹ to about 2.5*10⁻² mol L⁻¹g⁻¹s⁻¹.

In an embodiment, the carbon material is a carbon nano tube (CNT).

In an embodiment, the CNT is a single walled CNT (SWCNT).

In an embodiment, the ionic liquid is a hydrophobic ionic liquid.

In an embodiment, the ionic liquid comprises an aprotic solvent.

In an embodiment, the hydrophobic ionic liquid comprises a pyridinium containing ions.

In an embodiment, the composition contains less than 15% v/v of a protic solvent in the composition.

In an embodiment, the composition further comprises an indicator to detect the ROS.

In an embodiment, the composition is free of the protic solvent.

In an embodiment, the composition further comprises a solubilizing agent to increase diffusion coefficient of oxygen in the composition.

In an embodiment, the invention provides a system comprising: a) a reaction cell comprising a composition comprising an ionic liquid and a carbon material, b) a source of an electromagnetic radiation having wavelength in a range of about 150 nm to about 1100 nm, and c) a source of air comprising oxygen; wherein the source of the radiation and the reaction cell is arranged such that the radiation produced from the source catalyzes a reaction in the reaction cell to generate a reactive oxygen species (ROS) using the oxygen.

In an embodiment, the IL is a hydrophobic IL.

In an embodiment, the reaction cell comprises a stirrer.

In an embodiment, the stirrer is configured to dissolve the carbon material in the IL for a pre-determined time.

In an embodiment, the pre-determined time is in a range of 0 mins to 36 hours.

In an embodiment, the reaction cell contains a solubilizer that is configured to increase diffusion coefficient of the oxygen in the composition.

In an embodiment, the system further comprises a heating element to heat the composition to increase diffusion coefficient of the oxygen in the composition.

In an embodiment, an efficiency of the system to generate the ROS is about 2.5*10-4 mol L-1g-1s-1 to about 2.5*10-2 mol L-1g-1s-1.

In an embodiment, the ROS is superoxide.

In an embodiment, the system is configured for disinfection of water and air.

In an embodiment, the carbon material is a carbon nanotube (CNT).

In an embodiment, the CNT is a SWCNT.

In an embodiment, the source of the electromagnetic radiation is a UV lamp.

In an embodiment, the light intensity needed to create the right amount of ROS is about 1.5 W/m² (which is 0.11% of solar light intensity). It is only reasonable to assume that most of the useful ROS generating systems would need at least 1/10 of the above optimal value.

In an embodiment, the light intensity incident on the composition may be about 1.5 W/m².

In an embodiment, the light intensity incident on the composition may be about 0.11% of solar light intensity.

In an embodiment, the light intensity incident on the composition may be larger than a minimum value of about 0.15 W/m² such as 0.20 W/m², 0.25 W/m², 0.50 W/m², 1 W/m², 1.5 W/m², 2 W/m², 3 W/m², 4 W/m², 5 W/m² or more or within any range of the disclosed value.

The light intensity incident on the composition may be larger than a minimum value of about 0.011% of solar light intensity, such as about 0.015%, 0.020%, 0.030%, 0.050%, 0.1%, 0.15%, 0.2%, 0.5% or more of solar light intensity, or within any range of the disclosed value.

In an embodiment, the system further comprises a detection system to quantify the ROS.

In an embodiment, the detection system is a cyclic voltammetry technique.

In an embodiment, the invention provides a method comprising: irradiating a radiation having wavelength in a range of about 150 nm to about 1100 nm to a composition comprising an ionic liquid (IL) and a carbon material; introducing oxygen to the composition; catalyzing production of a ROS in the composition using the radiation; and producing the ROS.

In an embodiment, comprises preheating of the composition to increase diffusion coefficient of the oxygen in the composition.

In an embodiment, further comprises quantifying the ROS in situ.

In an embodiment, comprises measuring a ratio of I₂/I₁; wherein I₂ is a reduction reaction producing the ROS; wherein I₁ is an oxidation reaction consuming the ROS.

In an embodiment, a ratio of I₂/I₁ is more than 1.

In an embodiment, comprises controlling flow rate of the oxygen introduced in the composition.

In an embodiment, the invention provides a system comprising a chamber comprising a reaction chamber comprising a carbon material suspended in an ionic liquid, an inlet to allow passage of incoming air containing oxygen inside the chamber and an outlet to allow passage of outgoing air disinfected by a ROS produced inside the system by the reaction chamber in presence of a radiation having wavelength in a range of about 150 nm to about 1100 nm produced from a radiation source.

In an embodiment, the system is an air purifier.

In an embodiment, the chamber comprises a transparent surface.

In an embodiment, the chamber comprises an opaque surface.

In an embodiment, the chamber has both the transparent and the opaque surface.

In an embodiment, the radiation source is inside the chamber to illuminate the reaction chamber with the radiation to produce the ROS.

In an embodiment, the radiation source is outside the chamber and the radiation emitted by the radiation source is reflected inside the chamber to illuminate the reaction chamber to produce the ROS.

In an embodiment, the radiation source is sun, and the radiation is a solar radiation that illuminates the reaction chamber to produce the ROS.

In an embodiment, the system comprises a filter configured to filter the air before its reaction with the reaction cell to produce ROS.

In an embodiment, the system comprises a filter configured to filter the air coming out from the outlet of the system.

In an embodiment, the radiation source is a UV lamp.

In an embodiment, comprises a reflector configured to concentrate density of the radiation striking the reaction chamber.

In an embodiment, the surface of the chamber is curved in an angle, configured to concentrate the radiation reaching inside the reaction chamber.

In an embodiment, the system comprises a set of reflectors configured to reflect the radiation back and forth, to increase a time of reaction between the radiation and the reaction chamber.

In an embodiment, the system comprises a fan for circulation of the air inside the chamber.

In an embodiment, wherein the system comprises a louver.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The foregoing summary, as well as the following detailed description of various embodiments, is better understood when read in conjunction with the appended drawings. For the purposes of illustration, there is shown, in the drawings, exemplary embodiments; however, the presently disclosed subject matter is not limited to the specific methods and instrumentalities disclosed. In the drawings:

FIG. 1 shows a schematic for O₂⁻ generation by SWCNTs under photo illumination in IL and the in-situ O₂⁻ detection by electrochemical measurements. IL, ionic liquid; SWCNT, single-walled carbon nanotube.

FIG. 2 is an exemplary lab setup of the reaction and detection cell.

FIG. 3 depicts evaluation of the O2 generated from the cell: a) CV for the IL. b) normalized concentration of O2 in IL and SWCNTs (experimental group) and in three control groups. c) calculated concentration of O2. d) UV absorption spectra of the IL contained NBT²⁺.

FIG. 4 shows an electrochemical reaction cell setup and CV curves at various scan rates: (a) reaction cell setup. (b) and (c) CVs for IL in the absence of SWCNTs at room temperature and scan rates of 9, 36, 64, 81, and 100 mV/s respectively (d) and (e) CVs for IL in the presence of SWCNTs and IL suspension at room temperature and scan rates of 9, 36, 64, 81, and 100 mV/s. CV, cyclic voltammetry; IL, ionic liquid; SWCNT, single-walled carbon nanotube.

FIG. 5 shows Sustainability characterization for the SWCNTs/IL system and qualitative studies of O₂⁻ in the system. (a) FT-IR absorbance spectra for the suspension of IL and SWCNTs with UV light treatment after 0, 1, 2, and 3 h (hour). (b) The percentage of O₂⁻ remaining in the IL vs. time. (c) EPR spectrum of O₂⁻ generated by SWCNTs/IL suspension after 24 h of UV light treatment. (d) UV-visible absorption spectra of the SWCNTs/IL system containing NBT2 [symbol] . (e) Color change after 0, 2, 4, and 6 h for samples of #1. IL and ethanol; #2. IL, NBT²⁺ and ethanol (350 nm UV radiation); #3. IL, NBT2 [symbol] , and ethanol (dark). #4. IL, SWCNTs, and ethanol; #5. IL, NBT2 [symbol] , SWCNTs, and ethanol (350 nm UV radiation); #6. IL, NBT²⁺, SWCNTs, and ethanol (dark). EPR, electron paramagnetic resonance; FT-IR, Fourier-transform infrared; IL, ionic liquid; NBT, nitro blue tetrazolium; SWCNT, single-walled carbon nanotube; UV, ultraviolet.

FIG. 6 shows reduction and oxidation peak current density changes in (a) pure IL (dark), (b) pure IL (UV), (c) SWCNTs/IL (dark), (d) SWCNTs/IL (UV). (e) In-situ monitor of O₂⁻ levels in SWCNTs/IL and IL systems. (f) [O₂⁻] generated by SWCNTs vs. reaction time. IL, ionic liquid; SWCNT, single-walled carbon nanotube.

FIG. 7 depicts an exemplary example of a system according to an embodiment of the invention, configured to work as an air purifier. In the shown system, carbon fibers/CNT are shown as a carbon material.

### DETAILED DESCRIPTION

### Definitions and General Techniques

For simplicity and clarity of illustration, the drawing figures illustrate the general manner of construction, descriptions and details of well-known features and techniques that may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of the embodiments of the present disclosure. The same reference numerals, in different figures, denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The percentages mentioned herein should be construed as wt. % unless explicitly mentioned otherwise.

The following terms and phrases, unless otherwise indicated, shall be understood to have the following meanings.

The term, “ionic liquid (IL)” means a liquid that contains only ions. In the usual sense, this term includes all salts or mixed salts systems with melting points below about 150° C. (US20070006774A1). However, ionic liquids as used herein in some embodiments of the invention have melting points generally below room temperature and therefore termed as “room-temperature ionic liquids” (RTILs). In some cases, the ionic liquids are organic salts containing one or more cations that are typically ammonium, imidazolium, or pyridinium ions, etc. ILs also may be in combination with a variety of anions, ranging from simple inorganic ions (e.g., halides) to more complex organic species (e.g., triflate). ILs may have interaction capabilities such as: hydrogen bond acidity, hydrogen bond basicity, π-π, dipolar, and dispersion interactions. These interactions are directly related to the structures of the cationic/anionic moieties that comprise the IL.

ILs have almost negligible vapor pressures at room temperature, possess a wide range of viscosities, can be custom-synthesized to be miscible or immiscible with water and organic solvents, often have high thermal stability, and are capable of undergoing multiple solvation interactions with many types of molecules. U.S. Pat. No. 9,782,746B2 discloses polymeric ionic liquids.

A sub-class of ionic liquids are hydrophobic called as hydrophobic ionic liquids, and thus maintain consistent physical properties across a wide range of ambient humidity (U.S. Pat. No. 5,827,602). Few examples of hydrophobic ionic liquids are but not limited to 1-Butyl-1-methylpyrrolidinium bis(trifluoromethanesulfonyl)imide (BMPyrr NTf2), 1-Hexyl-3-methylimidazolium, 1-Octyl-3-methylimidazolium, 1-Methyl-1-(3-methoxypropyl)pyrrolidinium, 1-Ethyl-3-methylimidazolium, etc.

The term “aprotic solvent” as used herein refers to a solvent in which no hydrogen bonding takes place or they neither donate nor accept the proton (hydrogen).

The term “carbon material” is a generic term that means materials containing carbon atoms. Various carbon materials generally known in the art can be used as the carbon material according to the present invention such as graphite, carbon felt, carbon paper, carbon cloth, carbon black, a carbon nano tube (CNT), a carbon nano fiber (CNF), a vapor grown carbon fiber (VGCF), graphene, and the like. CNT as used in this invention could be single-walled carbon nanotube (SWCNT) or double-walled carbon nanotubes (DWCNT), multi-walled carbon nanotubes (MWCNT), bucky tubes, fullerene tubes, tubular fullerenes, graphite fibrils, and combinations thereof. However, CNT is not limited to these.

SWCNTs have excellent mechanical, electrical, thermal, and optical properties, which enable several applications in multiple fields, such as coatings and films, microelectronics, energy storage, biosensing, and water treatment. Further, CNT may be a mixture of metallic and semiconducting carbon nanotubes, or a high purity mixture with a specific chirality.

In an embodiment, carbon material includes porous carbon material. For example, the porous material is made by contacting a given carbon material with a moisture at high temperature for the formation of pore or by alkali activation of treating a given carbon material with a molten salt of alkaline metal hydroxide. (U.S. Pat. No. 9,656,870).

In an embodiment, the carbon material may be present in any shape or form such as particles, sheets, nano horns, tubular, fibrous, or a dendritic carbon nanostructure including a branched carbon-containing rod-shaped or annular material, etc.

The size of the carbon material as used in the present invention may be less than 1 μm., preferably less than 750 nm, or more preferably between 5 nm to 500 nm, for example, 5, 10, 15, 20, 30, 50, 100, 150, or 200 nm. The size may be between any two of the above values.

In an embodiment, carbon material may be functionalized such as described in U.S. Pat. No. 8,039,681B2, US20060159612, etc. known in the art.

The term “suspension” is used interchangeably with the term “dispersion”, and means “dispersion” as conventionally understood in the art. That is the carbon material exhibits an affinity for the dispersion medium so that the carbon material and the dispersion medium are mixed in equilibrium without forming an aggregate or a precipitate. As used herein, the suspension contains solid particles of nanocarbon material suspended/dispersed in the solvent.

In an embodiment of the invention, the term “suspension of nanocarbons” or “dispersion of nanocarbons” in the context of the present invention refers to fully exfoliated nanocarbons (i.e., individualized nanocarbons). For example, when the nanocarbons are carbon nanotubes, “fully exfoliated” refers to the fact that the starting carbon nanotubes, which are typically in the form of bundles of aggregated nanotubes, are separated into individual carbon nanotubes. Likewise, when the nanocarbons are graphene, “fully exfoliated” refers to the fact that the starting graphite material, which is composed of aggregated/stacked graphene planes, is separated into individual graphene planes.

In an embodiment, the “suspension of nanocarbons” or “dispersion of nanocarbons” in the context of the present invention is not limited to fully exfoliated nanocarbons (i.e., individualized nanocarbons). The nanotubes dispersed in the solvent may be in bundled form that may loosen later by itself in presence of the solvent.

In an embodiment, CNT aggregates may loosen during suspension, but may be confirmed in a state of CNT aggregates also (U.S. Ser. No. 11/345,600).

The solvent, as used, may be an ionic liquid, aprotic solvent, or the like. The solvent may be a degassed solvent or a degassed mixture of two or more solvents. Within the context of the present invention, a suspension or dispersion is fundamentally defined as a metastable system, i.e., a system in which phases will eventually separate after a certain amount of time.

The term “ROS” is an abbreviation of reactive oxygen species. It includes superoxide, oxygen radical, and other form species known in the art. In an embodiment of the invention, ROS is superoxide.

The term “stability” is used interchangeably with “lifetime”. As used herein ‘stability’ or ‘lifetime’ refers to a total time when a ROS remains in its original form in the solvent. The total time is calculated from the time of the formation of that ROS till the time of its consumption by other organic compounds or being broken down to other stable forms.

The lifetime of the ROS in accordance with the invention varies from about 10 secs to about 10 mins to about 36 hours to about 10 days or more. In an embodiment, lifetime of the ROS is about 10 mins, 30 mins, 60 mins, 1 hour, 2 hours, 3 hours, 4 hours, 5 hours, 6 hours, 7 hours, 8 hours, 9 hours, 10 hours, 15 hours or 20 hours or more. In an embodiment, lifetime is 1 day, 2 days, 3 days, 4 days, 5 days, 7 days, 10 days or more. The lifetime of the ROS could be any time falling within the mentioned ranges. In an embodiment of the invention, stability is with respect to the ROS, superoxide.

The term “half lifetime” or “half-lifetime” is defined is the time required for a quantity ROS to reduce to half of its initial value. In an embodiment of the invention, ROS has a half lifetime of about 10 secs to about 10 mins to about 36 hours to about 10 days or more. In an embodiment, lifetime of the ROS is about 10 mins, 30 mins, 60 mins, 2 hours, 3 hours, 4 hours, 5 hours, 6 hours, 7 hours, 8 hours, 9 hours, 10 hours, 15 hours or 20 hours or more. In an embodiment, lifetime is 1 day, 2 days, 3 days, 4 days, 5 days, 7 days, 10 days or more. In an embodiment, the half lifetime is 60 mins. In an embodiment of the invention, half lifetime is with respect to the ROS, superoxide.

The term, “ROS generation efficiency” is interchangeably used as “efficiency to generate a ROS”. It is defined as a molarity of the ROS produced in a reaction cell per unit time with respect to per gram of the carbon material suspended in the IL solution. In an embodiment, ROS generation efficiency could refer to generation of superoxide, hydroxyl radical or any other ROS species that could be formed in any embodiment of the invention.

The term “radiation” as used herein refers to electromagnetic rays having wavelength ranging UV light (about 100 nm to about 400 nm), visible rays (about 400 nm to about 800 nm) and infra-red radiation (about 800 nm to about 1200 nm). The radiation could be any wavelength falling between the above-mentioned ranges. Any light that has a wavelength in the range of 250 to 1100 nm could be used, such as near IR, visible light and UV light.

The term “source of the radiation” or “radiation source” or similar in meaning: Radiation used in the present invention could be derived from the solar source or a non-solar source. If the sun is the source of the radiation, then the radiation is addressed as a solar radiation.

A non-solar source of radiation that could be used in the invention is any known in the art such as but not limited to halogen lamp, mercury vapour lamps, incandescent lights, UV lamps, UV laser, or the like. In an embodiment of the invention, a specialized light source is unnecessary. A source that could produce a radiation that has a wavelength in the range of 250 to 1100 nm, which covers near IR, visible light and UV light, will work according to an embodiment of this invention.

The term “solubilizer” interchangeably used with “solubilizing agent” refers to an oxygen diffusion-enhancing compound that increases the availability of oxygen in the composition of the invention. It may be by influencing the molecular structure of IL and thereby promoting the movement (diffusion) of oxygen through the composition, or by chelating with oxygen to increase the diffusion of the oxygen in the composition. Increased dissolution of oxygen in the composition will allow more oxygen to be available for the reaction to produce ROS.

The term “disinfection” is a generic term referred to a process of cleaning the intended matter by killing microbes for example: bacteria, virus, etc. In an embodiment, the virus includes COVID-19 virus and its variants.

The term “sustainability” is interchangeably used with “reusability”. In an embodiment, the composition of the invention, more specifically IL, could be reused multiple times such as 2, 5, 10, 15, 20 50, 100 times or more for the generation and storage of ROS.

The term “indicator” is meant as a chemical probe that is useful for detecting global or selective reactive oxygen species, including superoxide, and which is further capable of providing a detectable or quantifiable signal. Indicators known in art to detect ROS especially superoxide could be used in the present invention. In an embodiment, indicator may comprise fluorescent material, for examples: DCFH, 2-[6-(4′-hydroxy)phenoxy-3H-xanthen-3-on-9-yl]benzoic acid (HPF) and 2-[6-(4′-amino)phenoxy-3H-xanthen-3-on-9-yl]benzoic acid (APF) are fluorescent probes for the detection of ROS (Setsukinai et al, 2003). US20170122954A1 provides a list of different indicator probes for ROS, which is incorporated herein by reference in its entirety.

In an embodiment, the invention relates to a composition comprising a carbon material and an ionic liquid, wherein the composition is configured to form reactive oxygen species in the presence of oxygen and a radiation having wavelengths in the range of about 150 nm to about 1100 nm.

In an embodiment, the composition contains the ROS, singlet oxygen. In another embodiment, the composition comprises the ROS, superoxide. In another embodiment the composition comprises a combination of superoxide and singlet oxygen. In other embodiments, composition herein can comprise one or more of singlet oxygen, superoxide, hydroxyl radical, hydroperoxy radical, or hydrogen trioxide. In additional embodiments, composition herein can comprise one or more ROS in combination with other reactive species which can include, for example, one or more organic radicals, such as organo-peroxyl radicals, acyl radicals, hydrocarbon radicals (e.g., methyl or other alkyl radicals), or carboxyl radicals. In additional embodiments, formulation herein can comprise one or more ROS in combination with other reactive species, such as radicals or trioxyorganoacids, such as trioxyacetic acid.

ILs as a type of better liquid environment, are good for stabilizing and storing ROS. In an embodiment, IL could also be used as an electrolyte in electrochemical measurements. The ionic liquid can be substantially free of water, organic solvent, and nitrogen-containing base.

In an embodiment, example of a hydrophobic ionic liquid is but not limited to BMPyrr NTf2. The hydrophobic ionic liquid can be used as a liquid environment and mixed with carbon material such as CNTs to form a suspension.

In an embodiment, ionic liquid dominated aprotic solution could be used.

In an embodiment, ionic liquid may contain a small amount of protic solvent. The protic solvent could be but not limited to ethanol. The small amount may be less than 25% v/v, less than 20% v/v, 10% v/v, 5% v/v or less.

In an embodiment, ionic liquid is completely free of protic solvent.

In an embodiment, ILs such as 1-butyl-1-methylpyrrolidinium bis(trifluoromethylsulfonyl)imide ([C4mpy] [NTf2]) can be used. In an embodiment, O2- could exist in IL such as [C4mpy] [NTf2] for over 9 h.

There is no hydrogen ion existing in the hydrophobic ionic liquid, therefore, ROS can last longer in the IL than in water. This allows a better chance to study and utilize the ROS.

Second, IL is especially useful due to its excellent electrochemical properties when used as an electrolyte.

In an embodiment, IL may not have any electrochemical or molecular structure change due to a long exposure time of the radiation such as but not limited to UV. The exposure time may be 24 hours, 2, days, 5 days, 15 days, 30 days, 60 days or more. In an embodiment, due to long exposure time of UV radiation, the electrochemical properties of IL may change about or less than 50%, less than 40%, less than 30%, less than 25%, less than 10% or less.

The amount of the carbon material added to the solvent containing IL for the suspension may be appropriately determined depending on the type of the carbon material and the solvent used for suspension of the carbon material.

In an embodiment, 0.0001% w/v to about 20% w/v of the carbon material could be suspended in the IL, preferably in the range of 0.001% w/v to 10% w/v, more preferably in the range of 0.001% w/v to 1% w/v. If the amount of the addition is less than 0.0001% w/v, the volume of the solvent may become disadvantageously too large. On the other hand, if the amount is more than 20% w/v, the suspension may disadvantageously have high viscosity, which makes the handling difficult.

In an embodiment, each mL of IL may contain about 0.001 wt % to about 10 wt % of the carbon material. In an embodiment, each ml of IL may have about 0.001 wt %, 0.01 wt %, 0.05 wt %, 0.1 wt %, 0.5 wt %, 1 wt %, 5 wt %, 10 wt % of the carbon material.

In an embodiment, it is possible that the carbon materials remain suspended in the IL for a predetermined time before production of ROS. According to the present invention the predetermined time could vary from 0 mins, 5 minutes, 10 minutes, 30 minutes, 60 mins, 5 hours, 10 hours, 1 day, 2 days, 3 days and more. The predetermined time may be between any two of the above values.

In an embodiment, liquid suspension consists of single wall carbon nanotubes (SWCNTs) and the Ionic liquids (ILs). In an embodiment, about 1.5 mL of IL containing 0.001 wt % single-walled carbon nanotube could continuously generate superoxide for at least 18 hours, and the half lifetime of superoxide in IL could last for over 60 hours.

In an embodiment, the single wall carbon nanotubes (SWCNTs) can generate superoxide in 350 nm or 975 nm UV light, or even simply under a solar light. In an application, the ROS generation from CNT could be activated by solar light. In an embodiment, a mixture type of semiconducting and metallic SWCNTs could generate ROS when using a 350 nm UV light.

In an embodiment, the invention provides a method for: generating superoxide comprising irradiating a radiation having wavelength in a range of about 150 nm to about 1100 nm to a composition comprising an ionic liquid (IL) and a carbon material introducing oxygen to the composition; catalysing production of a ROS in the composition using the radiation; and producing the ROS.

In an embodiment, efficiency of the system to generate the ROS is in a range of about 2.5*10⁻⁴ mol L⁻¹g⁻¹s⁻¹ to about 2.5*10⁻² mol L⁻¹g⁻¹s⁻¹. In an embodiment, efficiency could be in a range of 2.5*10⁻⁴ mol L⁻¹g⁻¹s⁻¹ to about 2.5*10⁻³ mol L⁻¹g⁻¹s⁻¹.

In an embodiment, the invention provides storing solution of ROS. In an embodiment, solution to store ROS comprises IL. In an embodiment, the storing solution of ROS is free of protic solution. In an embodiment, the IL is hydrophobic IL or aprotic or a mixture thereof.

In an embodiment, the ROS stored in the IL of the invention has a half-lifetime of about 10 secs to about 10 mins to about 36 hours to about 5 days to about 10 days or more. In an embodiment, the half lifetime of the ROS is 1 day, 2 days, 3 days, 4 days, 5 days, 7 days, 10 days, 20 days or more. In an embodiment, the half lifetime is 60 mins. In an embodiment of the invention, half lifetime is with respect to the ROS, superoxide.

In an embodiment, the ROS stored in the IL of the invention has a stability of about 10 secs to about 10 mins to about 36 hours to about 5 days to about 10 days or more. In an embodiment, the stability of the ROS is 1 day, 2 days, 3 days, 4 days, 5 days, 7 days, 10 days, 20 days or more.

In an embodiment, intensity of light source used in the invention was 7.5 W/m² (Hach Company 2184300), and it successfully triggered the generation of ROS. Considering the cross section of light source (45.6 cm²) is much bigger than the device (2 cm²), we estimate only less than 20% of irradiation, which is 1.5 W/m² successfully triggered the generation of ROS. In comparison, the intensity of solar light is roughly 1362 W/m² so, only 0.11% of solar intensity will be much more enough for a system to generate ROS. Further, it is reasonable to assume that most of the useful ROS generating systems would need at least about 1/10 of the above optimal value.

In an embodiment, the light intensity incident on the composition may be about 1.5 W/m².

In an embodiment, the light intensity incident on the composition may be larger than a minimum value of about 0.15 W/m², such as but not restricted to 0.20 W/m², 0.25 W/m², 0.50 W/m², 1 W/m², 1.5 W/m², 2 W/m², 3 W/m², 4 W/m², 5 W/m² or more.

In an embodiment, the light intensity incident on the composition may be about 0.11% of solar light intensity.

In an embodiment, the light intensity incident on the composition may be larger than a minimum value of about 0.011% of solar light intensity, such as but not restricted to 0.015%, 0.020%, 0.030%, 0.050%, 0.1%, 0.15%, 0.2%, 0.5% or more of solar light intensity.

In an embodiment of the invention, stability is with respect to the ROS, superoxide.

### Detection of Generated ROS

ROS can be detected by several techniques such as chromatography, spectrophotometry, and electrochemistry, with the help of different chemical scavengers or indicators. For example, 1O2 can be detected by furfuryl alcohol through high-performance liquid chromatography.

O₂⁻ could be detected using nitro blue tetrazolium salt (NBT²⁺), which reacts with O₂⁻ to produce monoformazan, a purple substance with optical absorption at 530 nm in water. This method is convenient and fast, but NBT²⁺ is vulnerable to other reactive substances and is unsuitable for low-level detection.

Terephthalic acid (TPA) is a convenient detector for HO·. The non-fluorescent TPA becomes fluorescent monohydroxy terephthalate upon reaction with HO·. The production can be excited at around 315 nm and emission is seen at 425 nm by using a fluorescence meter. This method is low-cost and fast, but TPA is also sensitive to UV light, which turns the sample fluorescent in a UV-dependent system.

Electrochemical methods can be used to selectively detect O₂⁻. O₂⁻ reacts with certain proteins, and these reacted proteins can be re-oxidized at a specific potential. However, only a limited number of sensitive proteins are available for O₂⁻ and additional preparation for the proteins is required.

The redox couple, O₂/O₂⁻, can be generated during cyclic voltammetry (CV). Wang et. al. [28] reported the oxidation peak for O₂⁻ was found near −1.2V vs. ferrocenium/ferrocene (Fc⁺/Fc) couple during the reverse CV scan at a Pt electrode. It is known the height of oxidation peak is related to the concentration of reactant, O₂⁻. This means that electrochemical methods can be used to study ROS generation in IL without introducing any additional indicators or quenchers.

In an embodiment, we propose an in-situ and accurate detection method for ROS, specifically O₂⁻, based on the principal of electrochemistry without needing additional indicators, equipment, or complicated sample preparation.

Considering the inert and low volatility nature, aprotic ionic liquid (IL) is an excellent solvent to study the properties of O₂⁻.

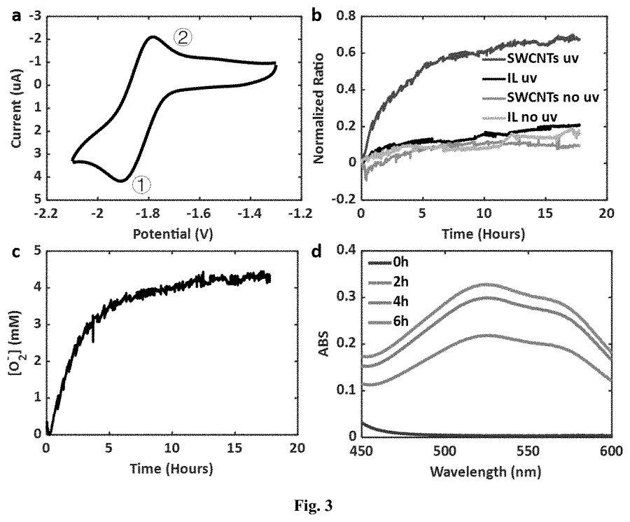

FIG. 2 provides an exemplary lab setup of the reactor for a typical ROS, superoxide (O₂⁻), assembled with an electrochemical system for detection.

The tiny white magnetic bar in the suspension is used to prevent the O₂⁻ from adsorbing on the working electrode and to prevent CNT particles from being aggregated. The cell is placed on a magnetic stirrer. The generation of O₂⁻ can be activated by a 350 nm Ultra-violet lamp which is placed on the left side of the reaction cell. The generation of the superoxide is evaluated by CV and chronoamperometry techniques based on a three-electrode system where the Au (gold) is used as a working electrode, and two platinum wires are used as the count electrode and reference electrode.

To confirm the production of O₂⁻ and determine the concentration of O₂⁻ ([O₂⁻]), a new detection method that combines the CV and chronoamperometry techniques was specifically designed. The detection is based on CVs scanning for the IL.

As shown in FIG. 3a, the peak \textcircled{1} represents the reduction reaction of O₂, where O₂ obtains electron to produce O₂⁻; peak \textcircled{2} represents oxidation reaction of O₂⁻, where O₂⁻ looses an electron to produce O₂. Based on the principal of the CV process, the peak height is correlated to the corresponding reactant. Namely, the peak height of peak \textcircled{1} and \textcircled{2} (represented by I₁ and I₂) are correlated to [O₂] and [O₂⁻], respectively. The ratio of I₂ to I₁ can be used to indicate the [O₂⁻] present in the cell. Thus, if the CNTs can produce extra O₂⁻, the ratio of I₂ to I₁ will increase.

The 17-hour reaction and detection experiment results are shown in FIG. 3b, where the ratios of I₂ to I₁ were normalized to ZERO. In FIG. 3b, one experimental group, containing SWCNTs and IL treated with UV light, and three control groups, that either did not contain SWCNTs or not treated with UV light or neither, were setup to evaluate the system.

Clearly, the relative ratio for the experimental group was much larger than that for the three control groups, which confirmed the role of SWCNTs and UV light and indicated the CNTs generated extra amounts of superoxide other than that generated during the CV process. The [O₂⁻] produced by SWCNTs was further determined by combining the CV and chronoamperometry techniques using electrochemical equations.

In an embodiment, [O₂⁻] related to the reaction time is shown in FIG. 3c. As a result, the total amount of O₂⁻ generated by SWCNTs was up to 4.45 mM in a mini-scale reactor that only contained 1.6 mL IL (BMPyrr NTf2) and 0.1 mg SWCNTs within 17 hours, which is comparable to germicidal levels for ROS (usually around 10 mM).

To further confirm the generation of O₂⁻ in our system, a traditional method was also used to detect the O₂⁻, which has been frequently used in many other publications. For this method, a kind of O₂⁻ indicator, Nitro blue tetrazolium chloride (NBT²⁺), was added into the cell. A purple product from the reaction of O₂⁻ and NBT²⁺ can absorb UV light at 530 nm. During this experiment, the cell was treated with 350 nm UV light and characterized with UV-VIS spectroscopy in 2 hours. As shown in FIG. 3d, the absorption intensities were clearly increased with longer UV irradiation at 530 nm. This result also proved the generation of O₂⁻ in the system.

### Photodynamic Effect of SWCNTs in IL

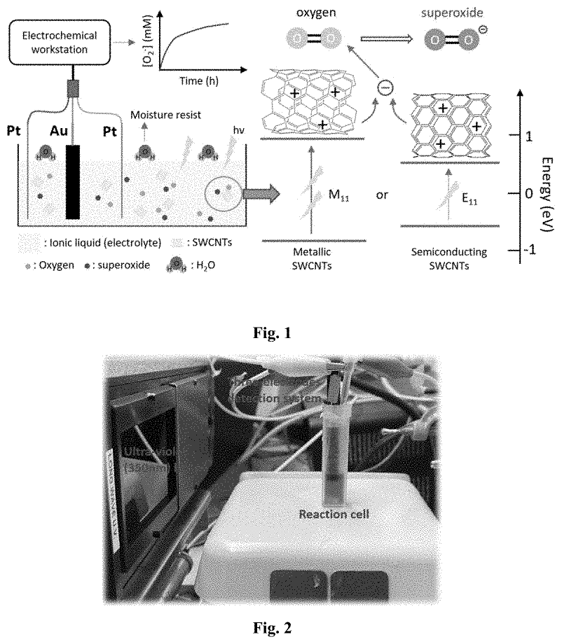

The term “photodynamic effect” refers to a process wherein the carbon material such as SWCNT is excited by photo illumination in a liquid media. A free electron is released by the photoexcited SWCNTs and captured by oxygen (O₂), and O₂⁻ is produced as shown in FIG. 1. O₂⁻ can further be reduced to HO· and H₂O₂ if a proton is present in the liquid media.

The efficiency of the photodynamic effect of SWCNTs is dependent upon the SWCNTs having either semiconducting or metallic properties, which is determined by the arrangement of carbon atoms (chirality). When SWCNTs are irradiated, the electrons in the valence band transition to the conduction band and can react with other active substances.

The specific ideal wavelength depends on the chirality of each SWCNT. As shown in FIG. 1, the semiconducting carbon nanotubes have a smaller optical transition gap and more optical transition paths than metallic CNTs. To overcome the band gaps of E₁₁ (FIG. 1), the irradiation wavelength is in the range of 900 nm-1400 nm and 550 nm-900 nm, respectively. In an embodiment, near infrared light about 975.5 nm could be used to excite SWCNTs and generate ROS.

Conversely, overcoming the bandgap of M₁₁ (FIG. 1) requires lower wavelength irradiation (350 nm-650 nm).

In an embodiment, the presence of metallic SWCNTs may relax the electrons away from semiconducting SWCNTs. The efficiency of the electron utilized by O₂ is higher than that relaxed by metallic SWCNTs, especially when semiconducting SWCNTs are dominant in the composition.

In an embodiment, regardless of chirality of SWCNT, a 350 nm UV light source could be used herein for the excitation. After being excited, SWCNTs can convert photon energy to fluorescence emission heat energy or trigger photodynamic reactions, which activate the adsorbed oxygen to produce ROS. The occurrence of photodynamic reactions is determined by the structure of SWCNTs.

In an embodiment, the photodynamic effect of SWCNTs in IL was investigated in [C₄mpy] [NTf₂]. The SWCNTs/IL system was illuminated with UV light to produce O₂⁻. The sustainability of the system was studied using UV-vis and Fourier-transform infrared (FT-IR) spectroscopy. Moreover, the generation of O₂⁻ was qualitatively confirmed using electron paramagnetic resonance (EPR) and UV-vis spectroscopy. To in-situ monitor the O₂⁻ concentration changes in the IL, a new detection method was established using CV. Finally, the concentration of O₂⁻ was quantitatively determined by conventional electrochemical methods. It is expected that this work will provide new insights into water and air disinfection fields.

### Materials and Methods

2.1. Materials

SWCNTs (>95%) were purchased from Sigma-Aldrich. 1-Butyl-1-methylpyrrolidinium bis(trifluoromethylsulfonyl)imide ([C4mpy][NTf2], 99.5%) was purchased from IOLITEC GmbH company. NBT chloride was purchased from Fisher scientific. Sodium dodecyl sulfate (SDS) was purchased from Sigma-Aldrich (ACS reagent, 99.0%). Deionized water (18.2 MU cm resistivity at 25° C.) was produced by Milli-Q Reference Water Purification System.

2.2. Optical Spectra Characterizations for SWCNTs by UV-Vis Spectroscopy

A UV-Vis spectrophotometer (GENESYS 150) was used to characterize the optical absorption of SWCNTs from 300 nm to 1100 nm. To prepare the suspension for UV-Vis spectroscopy, 3 mg of the as-received SWCNTs were added to 100 mL of 2 wt % SDS aqueous solution. The solution was then dispersed by probe sonication (Q500, Qsonica, LLC) in an ice bath for 30 min using a power of 100 Watts. After that, the dispersed SWCNTs in SDS aqueous solution were stabilized at room temperature and used as the analyte sample. A 100 mL of 2 wt % SDS aqueous solution without SWCNTs was also prepared and used as the benchmark.

2.3. Qualitative Determination of O₂⁻ by EPR

EPR spectroscopy was used to characterize the existence of O₂⁻ in the IL. Prior to EPR spectroscopy measurements, 0.1 mL of SWCNTs were added to 1.5 mL of [C4mpy] [NTf2] and stirred. Oxygen was then bubbled throughout the suspension and illuminated with UV light for 22 h. EPR studies were carried out using a Bruker EMXplus-9.5/2.7/P/L X-band continuous wave spectrometer (100 kHz field modulation, 3.2 G modulation amplitude at 45 dB) at 77 K. After the generation of O₂⁻, the solution was quickly transferred to an EPR sample tube and frozen in liquid N₂. No further adjustments were made before scanning.

2.4. Qualitative Determination of O2- by UV-Vis Spectroscopy Using NBT2+

To prepare the suspension for characterizing the O₂⁻ scavenger, 1 mg of as-received SWCNTs were added to 100 mL of ethanol to form a 10 mg/L suspension of ethanol and SWCNTs. The solution was then dispersed by probe sonication in an ice bath for 30 min using a power of 100 Watts. Finally, the suspension of ethanol and SWCNTs was obtained and stored in the dark until further use. NBT²⁺ salt was used to detect the O₂⁻. 1 mg NBT²⁺ was added to 1 mL IL and stirred for 30 min. The composition of analytes contained 0.1 mL NBT²⁺/IL suspension, 0.4 mL IL and 0.05 mL ethanol/SWCNTs suspension. Semimicro UV cuvettes were used to hold the samples. The reference sample did not contain any NBT²⁺. The samples for control groups did not contain any SWCNTs. The samples were illuminated with 350 nm UV light (6 Watts) and characterized using UV-Vis spectroscopy every 2 h. The details for the composition of each sample will be given in the discussion section.

2.5. Molecular Structure Characterization for IL Using FT-IR Spectroscopy

FT-IR absorption spectra of SWCNTs/IL suspension were recorded using a Varian 3100 Excalibur Series Spectrometer in the region of 400-2000 cm⁻¹. A single polyethylene ST-IR card supplied by Fisher Scientific was used for collecting spectra. A glass vial was filled with 6.05 mg/mL SWCNT concentrate in [C4mpy] [NTf₂] and stirred for at least 10 min. Then, the sample was irradiated with UV light for a total of 180 min using a Stratagene Stratalinker 1800 from Marshall Scientific while oxygen was simultaneously bubbled through the sample. Spectra were recorded at 60 min intervals.

2.6. Electrochemical Evaluations for Photodynamic Effect in SWCNTs/IL System

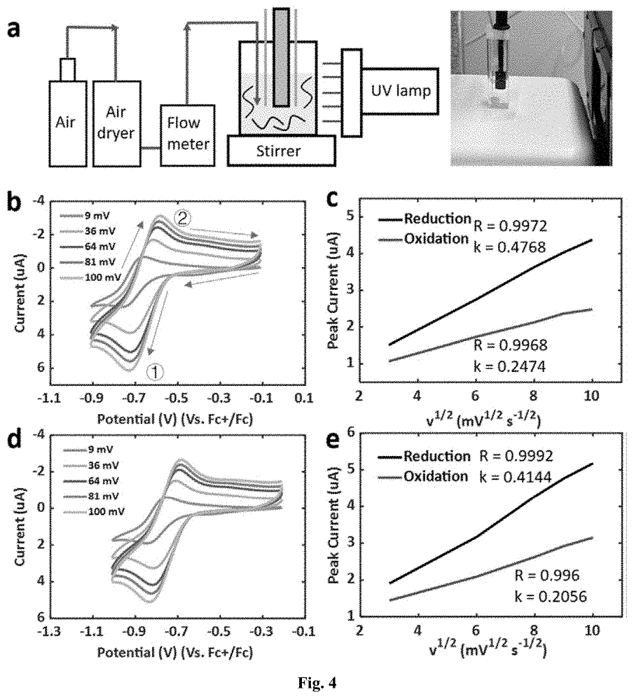

To prepare the stock suspension for electrochemical measurements, 10 mg of the as-received SWCNTs were added to 10 mL of IL to form a suspension. The suspension was then stirred in the dark for 36 h. The vial was sealed well during the stirring process to prevent the IL from absorbing moisture in the air. The dispersed suspension of SWCNTs and IL was obtained after the stirring and was stored in the dark until further use. The CV and CA measurements were performed by CH Instruments 660e. For CV measurements, a 2-mm gold electrode was used as the working electrode, and two platinum wires were used as the reference and counter electrode. For CA measurements, a 12.5-mm diameter gold microelectrode was used as the working electrode. The reference and counter electrodes were kept the same. A quartz cuvette cell (transmission range from 170 nm-2700 nm) was used as the electrochemical cell to hold the suspension. The entire setup of the experiments is shown in FIG. 4a.

It consisted of the gas flow system, EC system, and UV lamp. The air gas from a gas cylinder was dried by an air-drying column (Fisher scientific, Lot No. NC9760623). The flow rate was 100 sccm and controlled by a flow rate controller. A mini magnetic stirrer was added to the cell. A UV lamp (350 nm, 6 Watts) was placed toward the cell at a fixed position.

To prepare the electrolyte, 1.5 mL pure IL and 0.1 mL stock suspension were added to the cell. The concentration of SWCNTs in the IL was about 62.5 μg/ml. The cell was then dried in a vacuum oven at 60° C. overnight to remove moisture in the IL. Air was purged through the system prior to CV measurements; then, the electrochemical cell was stabilized for another 30 min. For all subsequent procedures, the airflow was purged to the surface of the IL rather than into the IL, and the electrolyte was stirred by a magnetic bar to avoid the aggregation of the SWCNTs. The power of the stirring was kept as low as possible to avoid any interference with the electrochemical process. Control group tests were performed by replacing the SWCNTs/IL suspension with pure IL and repeating the same procedures.

3. Results and Discussion

In this section, the sustainability of the SWCNTs/IL system under long-term UV light treatment will first be discussed in Section 3.1. The qualitative studies of O₂⁻ generated from SWCNTs in the IL is provided in Section 3.2. Then, a new method was established to in-situ monitor the O₂⁻ level in the IL system, which is introduced in Section 3.3. Finally, the concentration of O₂⁻ was quantitatively determined through conventional electrochemical methods in Section 3.4.

3.1. Sustainability Studies for SWCNTs/IL System by Optical Spectra Characterizations

To investigate the irradiation response of SWCNTs, the UV-VIS absorption spectra of SWCNTs from 300 nm to 1100 nm were obtained. It has been established that optical absorption spectra can identify SWCNT types. The absorptions at different wavelengths originate from the photo energy requirements for the electronic transitions of M₁₁, E₁₁, and E₂₂ (FIG. 1). From the spectra, the SWCNTs showed absorption at the UV range (300-400 nm) and had additional peaks at 587 nm and 1022 nm, which means irradiations at these ranges could be used to excite the SWCNTs. To take full advantage of all mixture types of SWCNTs, a 350 nm UV lamp was used for excitation. Moreover, combining these results with CV and CA data, it was determined that the 350 nm UV irradiation did not change the electrochemical properties of the IL (Section 3.3).

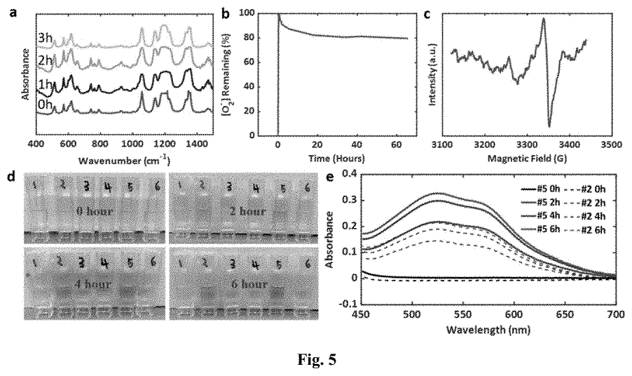

To further confirm that the IL had no molecular structural changes after UV illumination, FT-IR absorption spectra for the SWCNTs/IL suspension in the region 400-1500 cm⁻¹ was also obtained (FIG. 5a). The intense bands at 740 and 790 cm⁻¹ are contributed by S—N stretching and CF₃ bending vibrations, respectively, which aligned well with a previous report [35]. After 3 h of UV treatment, neither new bands appeared nor did any existing bands disappear, which indicated the molecular structure of the IL did not change due to irradiation. Further, this suggested that the IL could be reusable to some extent, showing the excellent sustainability of the system. The noise in the spectra was most likely caused by the presence of SWCNTs, which were black and had strong absorption across the full spectrum.

To investigate the long-term stability of O₂⁻ in the IL, O₂⁻ was specifically generated by CV. The experimental details are given in SI. The existence of CV generated O₂⁻ was confirmed using UV-vis spectroscopy where an absorbance peak occurred at 235 nm, which was consistent with other work [29]. The intensity of the absorbance at 235 nm was used to estimate the concentration of O₂⁻. As it shown in FIG. 5b, there was 79.7% of O₂⁻ still remaining in the IL after 65 h, this proved that the IL, [C4mpy] [NTf2], is an ideal media for storing O₂⁻ and its high sustainability for the system.

3.2. Qualitative Determinations for O₂⁻ Generated by Photodynamic Effect

To qualitatively confirm the system could generate O₂⁻ by photodynamic effect, EPR spectroscopy was used to characterize a UV-treated SWCNTs/IL suspension. As shown in FIG. 5c, the EPR spectrum of the UV-treated SWCNTs/IL suspension showed a single peak at around 3350 G, which was in agreement with other works [36,37]. Thus, the EPR spectra directly confirmed that O₂⁻ could be generated by SWCNTs via photodynamic effect.

The existence of O₂⁻ generated solely by SWCNTs was also confirmed by another conventional method using NBT²⁺ as an indicator. NBT²⁺ can react with O₂⁻ to generate a purple product, monoformazan. The formation of this product is indicated by an optical absorption peak around 530 nm, which can be detected by UV-vis spectroscopy [17]. Since the IL used here was aprotic, a small volume of ethanol was added to the reaction mixture as a proton source that is required to form the final product [38]. The addition of ethanol also improved the solubility of monoformazan compared to that in pure IL; this was especially beneficial since the mixture exhibited the better optical absorption results. As shown in FIG. 5d, the color of both #2 and #5 samples became purple with increasing UV illumination time. Both showed optical absorption at around 530 nm and the intensities of the absorptions increased with UV illumination time. All of these features were consistent with previously reported results [17]. The intensity of #5 was much larger than #2 (FIG. 2e), which confirmed the generation of superoxide by the photodynamic effect of SWCNTs, as only #5 contained SWCNTs. Samples #3 and #6 were placed in a dark room so that the SWCNTs could not utilize photon energy to generate O₂⁻, and no color change occurred. These results further confirmed the existence of O₂⁻ and demonstrated the correlation of O₂⁻ generated in the system and UV irradiation.

3.3. In-Situ Monitor for O₂⁻ Level in SWCNTs/IL System

To confirm the SWCNTs/IL system could be evaluated by electrochemical methods, CV measurements were performed in pure IL and in the SWCNTs/IL suspension at various scan rates. FIGS. 4b and 4d show typical CV curves obtained for the pure IL and the suspension of SWCNTs and IL at room temperature, respectively, where the curve has a forward reduction peak (labelled by \textcircled{1}) and a backward oxidation peak (labeled by \textcircled{2}). Ideally, when no other impurities were present, the O₂ was reduced to O₂⁻ in the reduction process, and the O₂⁻ was oxidized back to O₂ in the oxidation process. The presence of the oxidation peak (\textcircled{2}) suggested the existence of O₂⁻. The peak potential for O₂⁻ generation was −0.73 V (vs. Fc+/Fc). The CV curves in pure IL contained 0.01 M ferrocene. The features of the curve in pure IL were identical to that in the suspension, which indicated that the SWCNTs were not involved in the electrochemical process. The curve also aligns well with other works that used the same IL.

The CVs for the IL contained no SWCNTs or contained SWCNTs were performed at scan rates of 9, 36, 64, 81, and 100 mV/s. The results are provided in FIGS. 4b and d. It was found that the separation between the reduction and oxidation peak potentials changed with the scan rate (Table 1), which indicated the electrochemical generation of O₂⁻ was an irreversible process. The peak currents for the reduction and oxidation processes are proportional to the square root of the scan rate (FIGS. 4c and e), which confirmed the electrochemical process is under diffusion control.

This is also consistent with an irreversible electrochemical process [30,33]. These results proved that the SWCNTs/IL system could be evaluated and analyzed by electrochemical methods.

To establish an in-situ detection method for O₂⁻ in the SWCNTs/IL system, long-term, continuous CVs were performed while the system was simultaneously under UV irradiation. Three other samples were setup as control groups, including IL under UV treatment (IL UV), SWCNTs/IL without UV treatment (SWCNTs/IL no UV), and IL without UV treatment (IL no UV). Since the oxygen redox process in IL was under diffusion control, the oxidation peak current, iₚₒ, is positively correlated to three parameters, the concentration of O₂⁻ ([O₂⁻]), the diffusion coefficient of O₂⁻, and the scan rate of CVs [39]. The diffusion coefficient of O₂⁻ can be considered constant if the temperature is constant, thus, when using the same scan rate to perform the CVs, iₚₒ is proportional to [O₂⁻]. As such, the peak current density is an important factor used to estimate [O₂⁻].

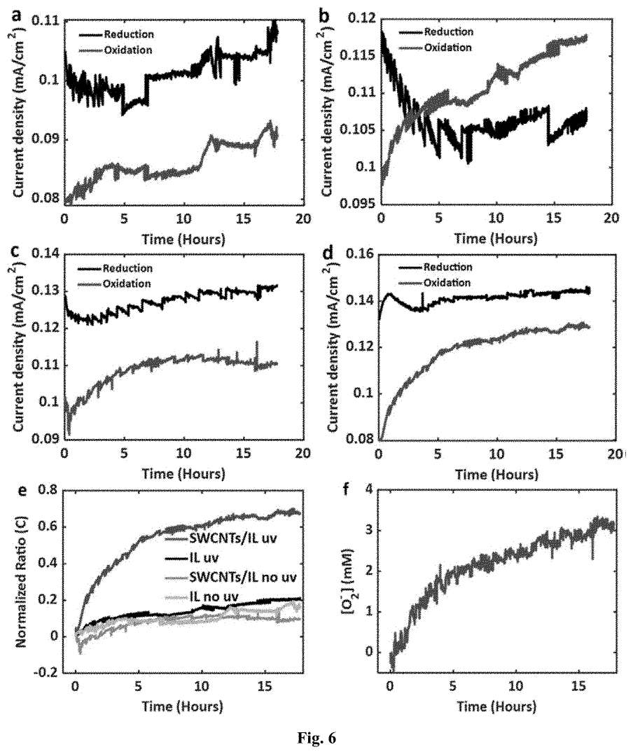

The kinetics of the photodynamic effect were explored using the change of peak current densities with reaction time. FIG. 6a shows the peak current density changes vs. the reaction time in pure IL electrolyte without UV illumination. It was found that both reduction and oxidation current densities exhibited a slight increase, which suggested that O₂ dissolved in the IL and was reduced to O₂⁻ in the CV process. This was consistent with previous reports [27,30,39]. When the pure IL electrolyte was illuminated by UV light (FIG. 4b), the reduction peak current densities of O₂ dramatically decreased while the oxidation current densities of O₂⁻ continued to increase. This was because the slight increase of temperature caused by UV illumination increased the diffusion coefficient of O₂ in the IL and facilitated the reduction reaction of O₂⁻ to produce more O₂⁻. The decrease of the concentration of O₂ ([O₂]) in the IL leads the reduction of current densities to decrease during the first half of scanning. We believe that the ability of O₂ from air to dissolve in IL is weak and slow. Though the air was purging to the surface of IL, the local [O₂] near the working electrode was decreased in the first 5 h. Thus, O₂ was rapidly consumed, and O₂⁻ was rapidly generated in the first 5 h. Later, the consumption and compensation of O₂ reached dynamic equilibrium. The reduction current densities no longer decreased, and the increased rate of the oxidation current densities was also smaller than the first 5 h. This indicated that the [O₂] was stable, and the increased rate of O₂⁻ became slower after 5 h.

When SWCNTs were added to the IL in the dark (FIG. 6c), the reduction and oxidation current densities exhibited a similar increase to that in FIG. 6a. This indicated that the presence of SWCNTs in the electrolyte did not change the electrochemical system. This was consistent with previous CV experiments using various scan rates (FIG. 4). When the electrolyte suspension was treated with 350 nm UV light (FIG. 6d), the reduction current densities only decreased for a short period of time (1-3 h) and then slightly increased. This was similar to the case in FIG. 6b and proved the repeatability of the different experiments. The oxidation current densities show a significant increase in FIG. 6d, which suggests the [O₂⁻] was greatly increased by the photodynamic effect.

Herein we have established a new method to in-situ monitor the relative O₂⁻ levels in the SWCNTs/IL system. Based on the fundamentals of electrochemistry, peak current is correlated to the concentration of reactant [33]. Here, the ratio of oxidation peak current to reduction peak current (R=iₚₒ/iₚᵣ) was used to evaluate the generation of O₂⁻ from the photodynamic effect of SWCNTs. iₚₒ is correlated to [O₂⁻] in the IL overall, and ipr is correlated to the [O₂] that was involved in the reduction reaction where a small amount of O₂⁻ was chemically generated. Thus, the defined R could be used to evaluate the [O₂⁻] distinctly generated by SWCNTs in the system and excluding any O₂⁻ produced from electrochemical reaction. Considering the increase of O₂⁻ led to the oxidation reaction during CV to produce O₂, such a contribution of O₂ would likewise increase ipr during the reduction process. Thus, a constant ipr, when t=0, was used for calculations and is represented by iₚᵣ (0); iₚₒ is given as a function of reaction time and is represented by iₚₒ (t). As a result, R(t) is given by Eq. (1).

R(t)=iₚₒ(t)/iₚᵣ(0) (1)

To make comparisons among control groups and experimental groups, R(t) was normalized by Eq. (2):

C(t)=(R(t)−R(0))/R(0) (2)

Where C(t) represented the relative [O₂⁻] generated by SWCNTs as a function of reaction time. The in-situ monitoring results are provided in FIG. 6e. The overall growth trend for the red curve (SWCNTs/IL with UV light) was much larger than the other three curves. This confirmed the generation of O₂⁻ by SWCNTs when illuminated with UV light since the normalized ratio did not increase when UV illumination (‘SWCNTs/IL no UV’) or SWCNTs (‘IL UV’) were absent. Besides, it has been reported that metal catalyst from CNTs was involved in the formation of ROS, such as Nickle [17]. However, the ‘SWCNTs/IL no UV’ curve was on similar level as the other two control groups. It indicated that the SWCNTs/IL system could not generate O₂⁻ in absence of UV light. This was consistent with the result from NBT²⁺ characterization and ruled out the possibility that the catalyst metal from SWCNTs was involved in the reaction. In fact, the sample used in this work was highly purified SWCNTs. After 100-fold dilution, the concentrations of other impurities were even lower. Thus, the catalyst metal has negligible impact on the system. Comparing the ‘IL UV’ curve with the ‘IL no UV’ curve, it was found that UV illumination slightly facilitated the reduction reaction of O₂. The slight increase of temperature caused by the UV illumination increased the diffusion coefficient of O₂, which was consistent with the current density analysis discussed previously. When both SWCNTs and UV illumination existed (‘SWCNTs UV’ curve), O₂⁻ was quickly generated, and the [O₂⁻] under these conditions was much larger than that of control groups. This demonstrated the photodynamic effect of SWCNTs when the system was illuminated with UV light. The newly established method was able to in-situ monitor the O₂⁻ level in the SWCNTs/IL or pure IL system.

3.4. Quantitative Determination of O2- by Conventional Electrochemical Measurements

[O₂⁻] was quantitatively determined by both CV and CA measurements using a 2-mm and 12.5 mm gold microelectrode, respectively, via conventional electrochemical calculations. According to Hayyan et al. and Katayama et al. [39,40], for an irreversible, one-step, and one-electron electrochemical reaction (O₂+e [symbol] O₂⁻), the peak current and potential from CV measurements are given by the following Eqs. (3)-(5) [33]:

$${i}_{pr} = ( 2.99 * {10}^{5} ) {α}^{1 / 2} {AC}_{o} {D}_{o}^{1 / 2} {p}^{1 / 2} \qquad ( 3 )$$

$$\vert {E}_{p} - {E}_{p / 2} \vert = \frac{1.857 RT}{α F} \qquad ( 4 )$$

Where α is charge transfer coefficient; A is the surface area of the working electrode in cm²; Cₒ is the bulk concentration of O₂ in mol/ml; Dₒ is diffusion coefficient of O₂ in cm²/s; n is scan rate in V/s; Eₚ is the peak potential in V; E_{p/2} is the half-peak potential in V; R is the universal gas constant in J(mol. K); T is the absolute temperature in K; F is Faraday's constant, 96,485 C/mol.

TABLE 1
Peak-to-peak potential separation at various scan rates.

| Scan rate | \|Eₚᵣ − Eₚₒ\| (mV) | | \|Eₚ − E_{p/2}\| (mV) | \|Eₚ − E_{p/2}\| (mV) |
|---|---|---|---|---|
| (mV/s) | IL | IL + SWCNTs | IL | IL + SWCNTs |
| 9 | 103 | 96 | 79 | 88 |
| 36 | 111 | 106 | 103 | 100 |
| 64 | 134 | 128 | 103 | 100 |
| 81 | 139 | 138 | 101 | 104 |
| 100 | 151 | 151 | 101 | 103 |

|Eₚᵣ − Eₚₒ|: The difference between reduction peak and oxidation peak potential.
|Eₚ − E_{p/2}|: The difference between reduction peak and half reduction peak potential.

From CA measurements, the steady-state current (iss) was obtained and given as follows:

iₛₛ=4nFDₒCₒrₒ (5)

Where n is the number of electrons and ro is the radius of the electrode. Consequently, Co and Do can be solved using Eqs. (3)-(5). As shown in Table 2, [O₂] was smaller in the suspension than in pure IL, but the diffusion coefficient of O₂ for the suspension and IL were inversely related. This suggested SWCNTs absorbed a portion of O₂ molecules that dissolved in the IL. During the forward voltage scan, the dissolved O₂ molecules diffused to, and were reduced at, the working electrode, considering this, SWCNTs might also be driven to the electrode. The evidence suggested SWCNT aggregation occurred during long-term CV measurements. The conductive and tiny SWCNTs may diffuse faster than O₂ when driven by voltage. As a result, the diffusion coefficient of O₂ increases.

TABLE 2
Concentration of O₂ (Cₒ), diffusion coefficient of
O₂ (Dₒ), steady-state current (iₛₛ), charge transfer
coefficient (a) in suspension, and pure IL.

| | Cₒ (mmol) | Dₒ (10⁻⁹ m²/s) | α | iₛₛ (nÅ) |
|---|---|---|---|---|
| Suspension | 0.43 | 2.8 | 0.36 | 2.897 |
| IL | 1.33 | 0.33 | 0.44 | 1.070 |

If Eq. (3) is applied to both reduction and oxidation processes and substituted into R, Eq. (6) is given by:

R=iₚₒ/iₚᵣ=CₓDₓ/CₒDₒ (6)

Where Cₓ is [O₂⁻] and Dₓ is the diffusion coefficient of O₂⁻.

The diffusion coefficient of O2- in this IL is constant and was taken from the literature [39]. In Eq. (6), Co and Do can be obtained by Eqs. (1) and (3), which is given in Table 2. Co was assumed to be constant since the air was continuously purged into the electrochemical cell. Dr and Do were also assumed to be constant, as they were only dependent on temperature. Thus, using Eq. (6), the [O2-] for all four samples, SWCNTs/IL UV, IL UV, SWCNTs/IL no UV, and IL no UV, was determined. After calculations, the initial [O2-] was normalized to zero mM at time zero. The [O2-] generated by SWCNTs changed with reaction time and is given in FIG. 6f, where the electrochemically generated [O2-] has already been subtracted.

Based on FIG. 6f, the SWCNTs/IL system generated 4.11 mM of O₂⁻ within 17 h. since the CV process could generate a small amount of O₂⁻. After subtracting the partial concentration of O₂⁻ from CV, the total amount of O₂⁻ from photodynamic effect was determined to be 3.19 mM. In the first 60 min, the SWCNTs/IL system produced 190 mM of O₂⁻, which was more than six times greater than a recently reported ROS generator that produced 29.62 mM of O₂⁻ in 60 min [3]. Meanwhile, the reaction rate constant (k₀=d[O₂⁻]/dt) for the photodynamic effect was determined to be 4.99*10⁻⁸ mol L⁻¹s⁻¹. This exhibited a high generation efficiency of O₂⁻ in this system. Taking the weight of SWCNTs (62.5 mg/ml) into consideration, k₀ was determined to be 4.99*10⁻⁴ mol L⁻¹g⁻¹s⁻¹. The generated O₂⁻ herein was still excessive with respect to the germicidal levels of ROS. Given the small scale of this system, which only contained 1.6 mL IL and 0.1 mg SWCNTs, it is expected the generation efficiency could be improved by enlarging the scale of the system adding more SWCNTs and adjusting the wavelength of illumination. The low-level detection of [O₂⁻] by electrochemical techniques could provide feedback for those improvements and help optimize the system in future research.

Conclusion

This study illustrates the newly developed SWCNTs/IL ([C4mpy][NTf₂]) suspension is a sustainable system to efficiently produce and store ROS for long-term. The photodynamic effect of SWCNTs could enable high-yield O₂⁻ generation in non-aqueous IL media under UV illumination. The generation of O₂⁻ by SWCNTs in the IL was qualitatively confirmed by UV-vis and EPR spectroscopy, as well as electrochemical measurements. The IL was confirmed to have no structural changes after long-term UV light treatment. Based on CV measurements, the SWCNTs/IL system works with electrochemical methods, which enable in-situ monitoring of O₂. To the best of our knowledge, this is the first time that generating O₂⁻ by SWCNTs in IL without interference from water, and in-situ monitored O₂⁻ levels by CV has been reported. This new suspension system could be used as an effective generator and sustainable storage media for O₂⁻, as well as a real-time in-situ monitor for the low-level detection of O₂⁻.

In an embodiment, the present invention provides a disinfection technology for public health, using carbon materials to kill most of bacteria or virus, ensure cleanliness and safety and reduce toxicity level.

In an embodiment, the system could also be used as a starting point for generating O₂⁻ using other materials in the future. It is expected that this work will help elucidate the kinetics of ROS generation using carbon materials and pave the way for their applications in air and water disinfection and O₂⁻ sensitive chemical sensors.

Application

The technology can potentially be used in disinfection for public health. We can either use the technology to produce the superoxide, which is a kind of efficient disinfectant to kill bacteria or virus or put the carbon nanomaterials on any surface where the public spaces need to be cleaned.

As long as there is irradiation, the disinfectant will be produced. Due to the excitation range of the irradiation being very large, the production still works even at night.

The corresponding new detection and storage method for the ROS will also be developed to solve the detection difficulties of the ROS by traditional methods and the fast decay of the ROS. The generation efficiency of the ROS can be measured by electrochemical measurements such as cyclic voltammetry, chronoamperometry. The ROS can be stored in a kind of ionic liquid or ionic liquid dominated aprotic solution.

Considering the superoxide is the main disinfection substance, the anti-microbial effect of the current invention can last for at least 60 hours, even after the light source is off. The anti-microbial duration can be optimized in the future by engineering the IL/carbon surface, and designing electrochemical cells for stable and sustainable ROS generation and anti-microbial reaction.

In an embodiment, ROS produced may have various industrial or domestic oxidation, clean up and disinfection applications, such as but not limited to water treatment. In an embodiment, the system has application in air purification.

Air Purifier

In an embodiment, the invention provides a system comprising a chamber comprising a reaction chamber comprising a carbon material suspended in an ionic liquid, a duct having an inlet to allow passage of air containing oxygen inside the chamber and an outlet chamber to allow passage of the air disinfected by a ROS produced inside the system by the reaction chamber in presence of a radiation having wavelength in a range of about 150 nm to about 1100 nm produced from a radiation source.

In an embodiment, purification of the air at least includes the destruction of airborne pathogens. It is contemplated and understood that the air purifier system may be independent from other air conditioning systems or may be an integral part of such a system. For example, the air purifier system may be an integral part of a heating, ventilation, and air conditioning (HVAC) system also known to control air temperature and/or air humidity. It is further contemplated that the air purifier system may be installed as a unit into pre-existing duct work of the HVAC system.

In an embodiment, the air purifier system may include a chamber, a radiation assembly, a fan, a filter. During operation, the air (in a non-purified state) flows inside the chamber via the inlet into the reaction chamber, and axially out through the outlet portion (in a purified state).

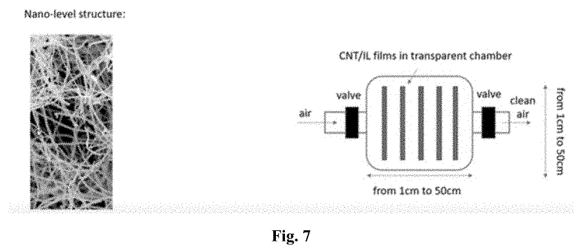

The chamber contains a reaction chamber located between the inlet and outlet portions. In an embodiment, the chamber may be of any shape such as rectangular, square, etc. The reaction chamber may be arranged in columns (as shown in FIG. 7), diagonally or in any manner as per the need and aesthetic appeal of the person skilled in the art. Similarly, the reaction chamber containing the carbon material and the IL could be of any shape.

In one example, the radiation source is located in the chamber. In an embodiment, the radiation source is located inside the reaction chamber.

In an embodiment, the filter may be located at the inlet portion and upstream from the reaction chamber. The fan may be located upstream of the reaction chamber and may be near the inlet to increase circulation of air.

In an embodiment, the filter is adapted to reduce, or eliminate the flow of particulates in the air, before the air reaches the reaction chamber.

In an embodiment, the louver is adapted to divert the flow pattern of the incoming air to that of a spiraling pattern for optimizing the performance of the ROS in the destruction of pathogens. It is contemplated and understood that the filter may be located at the outlet portion of the chamber, or an additional filter may be located at the outlet portion.

In one example, the face of the chamber may be reflective for the reflection of radiation produced by a source (i.e., energy or rays) back into the reaction chamber to optimize performance of the reaction chamber in the destruction of the pathogens. Similarly, each airfoil of the fan may include a reflective surface that faces, at least in-part, toward the reaction chamber. In operation, the radiation may be reflected off of the angled, reflective surfaces of each airfoil and back into the reaction chamber at various angles for improved distribution of the radiation for improved production of ROS and destruction of the pathogens. In an embodiment, the reflective surfaces may be made of polished aluminum, polished stainless steel, or any other material or composition capable of reflecting the radiation in particular ultraviolet rays.

In one embodiment, the radiation source is located in the chamber or is proximate to the inlet portion and/or louver or is centered to the centerline. It is contemplated and understood that the radiation source may be orientated elsewhere and may be located outside of the chamber provided the radiation is efficiently emitted into the chamber into the reaction chamber.

In one embodiment, the fan may be a passive fan employed to rotate (i.e., like windmill) via the air flowing through it. An electrically driven fan may be located elsewhere in the system, not exposed to the ultraviolet light, and to induce the flow of air.

Advantages and benefits of the present disclosure include an air purifier system capable of efficiently destroying pathogens.

While the present disclosure is described with reference to exemplary embodiments, it will be understood by those skilled in the art that various changes may be made and equivalents may be substituted without departing from the scope of the present disclosure. In addition, various modifications may be applied to adapt the teachings of the present disclosure to particular situations, applications, and/or materials, without departing from the essential scope thereof. The present disclosure is thus not limited to the particular examples disclosed herein, but includes all embodiments falling within the scope of the appended claims.

Differences Over Eleclean

Eleclean is a technology company that focuses on disinfecting areas. One of their products is to use electro-oxidation method to generate ROS, including hydroxyl radical, hydrogen peroxide and superoxide radical. The idea of using the ROS confirms the fundamental of the current invention from theory. However, most ROS, like superoxide and hydroxyl radical, will decay in the water within a few microseconds. They will turn into the more stabilized substance like hydrogen peroxide, which is less reactive than before. Due to water evaporation, this technique could not be used for long-term and heavy-duty air cleaning work. Compared with the Eleclean, the current invention should have more efficient and powerful disinfection abilities, as we are using a different liquid environment for ROS to exist. Moreover, the Eleclean uses consumable batteries to drive the electro-oxidation, while the current invention uses solar light to activate the disinfection function. Since the high stability of ionic liquid, our technique can be used as an air-cleaning component in a regular ventilation system for long-term operation. It is apparent that the current invention is a more environmentally friendly and less-costly technology.

All references, including granted patents and patent application publications, referred herein are incorporated herein by reference in their entirety.

1.-60. (canceled)

61. A composition comprising:
(a) a carbon material and
(b) an ionic liquid (IL);
wherein the composition produces a reactive species oxygen (ROS) when the composition is exposed to oxygen and an electromagnetic (EM) radiation illuminating the composition from a radiation source external to the composition;
wherein the EM radiation has a wavelength from about 150 nm to about 1100 nm.

62. The composition of claim 61, wherein the ROS is superoxide.

63. The composition of claim 61, wherein the carbon material is a carbon nanotube.

64. The composition of claim 61, wherein about 0.001% w/v to about 1% w/v of the carbon material is suspended in the IL.

65. The composition of claim 62, wherein about 85% of superoxide generated by the composition has a stability for at least 75 hours in the IL.

66. The composition of claim 62, wherein a half lifetime of the superoxide in the IL is up to 200 hours.

67. The composition of claim 62, wherein a generation efficiency of the superoxide by the composition is in a range of about 2.5*10⁻⁴ mol L⁻¹g⁻¹s⁻¹ to about 2.5*10⁻² mol L⁻¹g⁻¹s⁻¹.

68. The composition of claim 61, wherein the ionic liquid is a hydrophobic ionic liquid.

69. The composition of claim 61, wherein the ionic liquid comprises an aprotic solvent.

70. The composition of claim 68, wherein an intensity of the EM radiation incident on the composition is about 1.5 W/m².

71. The composition of claim 61, wherein the composition is free of the protic solvent.

72. A system comprising the composition of claim 61 and the radiation source.

73. The system of claim 72, wherein the system comprises a stirrer.

74. The system of claim 72, further comprises a detection system to quantify the ROS

75. A method comprising obtaining the composition of claim 61; exposing the composition to oxygen and the electromagnetic (EM) radiation having the wavelength from about 150 nm to about 1100 nm; and producing the reactive species oxygen (ROS).

76. The method of claim 75, further comprises measuring a ratio of I₂/I₁; wherein I₂ is a reduction reaction producing the ROS; wherein I₁ is an oxidation reaction consuming the ROS.

77. The method of claim 76, wherein a ratio of I₂/I₁ is more than 1.

79. The system of claim 72, wherein the composition is enclosed within a chamber.

80. The system of claim 72, wherein an intensity of the EM radiation incident on the composition is at least about 0.011% of a solar light intensity.

81. The system of claim 72, wherein the system is configured to disinfect air.
